(19) United States
(12) Patent Application Publication
(10) Pub. No.: US 2021/0015996 A1
(43) Pub. Date: Jan. 21, 2021

(54) METHOD AND DEVICES FOR DELIVERING INSULIN

(71) Applicant: Roche Diabetes Care, Inc., Indianapolis (IN)

(72) Inventors: Sven Winheim, Mannheim (DE); Oliver Kreidemacher, Mannheim (DE); Hans List, Mannheim (DE)

(21) Appl. No.: 17/065,385

(22) Filed: Oct. 7, 2020

Related U.S. Application Data
(63) Continuation of application No. PCT/EP2019/058758, filed on Apr. 8, 2019.

(30) Foreign Application Priority Data
Apr. 9, 2018 (EP) 18 166 314.7

(51) Int. Cl. A61M 5/145

(52) U.S. Cl. CPC A61M 5/1452; A61M 2205/3365; A61M 2205/103

(57) ABSTRACT

An inventive drive system for an insulin pump includes a motor configured to rotate at a predetermined revolution speed. It also includes a gear box configured for acting on a piston of the insulin pump to thereby convert a rotation of the motor into a continuous linear motion of the piston via a threaded rod mechanically coupled to the piston. The continuous linear motion of the piston determines a basal rate of insulin delivery. Additionally, a mechanical displacer is provided to act on the piston by adding a mechanical displacement of the threaded rod to the continuous linear motion of the piston, such as during a time then a bolus of insulin is to be delivered in addition to the basal rate. The threaded rod is mechanically coupled to the piston independent of the basal rate. An insulin pump and a method for driving an insulin pump are also disclosed.

### RELATED APPLICATIONS

This application is a continuation of PCT/EP2019/058758, filed Apr. 8, 2019, which claims priority to EP 18 166 314.7, filed Apr. 9, 2018, the entire disclosures of both of which are hereby incorporated herein by reference.

### BACKGROUND

This disclosure relates to a drive system for an insulin pump, to an insulin pump comprising the drive system and to a method for driving an insulin pump. The method and devices according to this disclosure may mainly be used for delivering insulin to a user. This disclosure may both be applied in the field of home care as well as in the field of professional care, such as in hospitals. Other applications are generally feasible.

Delivering medicine to a user, specifically insulin delivery, plays an important role in the prevention and treatment of diseases, in particular in the treatment of diabetes mellitus. Besides by using injection pens or syringes, insulin delivery may specifically be performed by using insulin pumps.

In general, electronically or electromechanically driven pumps require complex electronic components for controlling and monitoring. Such pumps are generally prone to malfunction or failure due to failure of electronic or electromechanical components. Specifically, in the field of delivering medicine, such as insulin, exact administration and control of the amount of medicine is critical. Consequently, error resistant insulin pumps operating without an electronic control are desirable. Although mechanical pumps operating without electronic control are generally known from the art and despite the advantages of state of the art pumps for delivering insulin, several technical challenges remain.

In particular, a user is generally required to wear the insulin pump on his or her body at all times, thus leading to a preferably small and compact construction of the insulin pump and its components. However, common pumps for delivering medicine, such as, for example, insulin, comprise a plurality of medicine reservoirs. As an example, fluid delivery devices are disclosed in WO2011/046950 A1. The fluid delivery device comprises a housing having a fluid reservoir. A needle is in fluid communication with the fluid reservoir in an engaged position and out of fluid communication with the fluid reservoir in armed and storage positions. A proximal end of a biasing member is coupled to the housing and a distal end of the biasing member is configured to deliver a force to the fluid reservoir. A piston member extends through the biasing member and is coupled to the distal end of the biasing member. The piston member is fixed with respect to the housing in a locked position such that the biasing member does not deliver the force to the fluid reservoir and the piston member is moveable with respect to the housing in a released position such that the biasing member delivers the force to the fluid reservoir. Transitioning the needle from the storage position to the armed position transitions the piston from the locked position to the released position.

WO2007/108969 discloses a disposable infusion device comprising a base arranged to adhere to a patient's skin, a cannula arranged to extend from the base to beneath the patient's skin to deliver a liquid medicament to the patient, and a source arranged to provide the cannula with a liquid medicament. The device further includes an actuator that actuates the source to provide the liquid medicament to the cannula. The source is arranged to provide, with each actuation, a fixed volume of medicament to the cannula. A control sets the fixed volume.

U.S. Publication No. 2003/009133 A1 describes a pump system for an infusion system including a linear drive which minimizes the space occupied by the pump components in a portable housing. A motor and a motor drive shaft are arranged in parallel with, and adjacent to a syringe and lead screw. A gear box connects the drive shaft and lead screw to transfer rotational movements between them. A piston driving member, such as a cone or drive nut converts the rotational movement of the lead screw into linear motion of a syringe piston. Sensors detect when the piston or cone is in a “home” position and in an “end” position, respectively. Optionally, a proximity sensor is used to ensure that the cone and the piston are abutting during dispensing. Alternatively, a clamping member selectively clamps the lead screw against linear motion in at least a dispensing direction.

WO 2015/104412 A1 describes a transmission comprising a first gear wheel and a second gear wheel with a threaded bore, and a threaded non-rotationally arranged rod in threaded engagement with the threaded bore, rotation of the second gear wheel thereby providing axial movement of the rod. The first and second gear wheel are arranged in a common plane and in rotational engagement with each other, wherein the combined second gear wheel and rod are arranged to pivot corresponding to a center point defined by the intersection of the rod axis and the common plane, whereby the rod, with the gear wheels in engagement, can be arranged out of alignment with the first gear wheel axis.

U.S. Publication No. 2012/179112 A1 describes a medicament delivery device comprising a housing for holding a medicament cartridge, a piston rod and a drive. The medicament cartridge has a medicament outlet and a bung moveable axially along the medicament cartridge for dispensing a medicament, the piston rod has a plunger for moving the bung and a lead member telescopically coupled to the plunger that may be driven by the drive to extend or retract the piston rod. Additionally, the device comprises a linkage coupled between the plunger and an anchorage and a drive member telescopically coupled to the lead member. The drive is operative to rotate the drive member to telescopically move the lead member relative to the drive member whereby the plunger is moved relative to the lead member by way of the linkage.

EP 1 195 172 A2 discloses an automatic injection device having piston holders holding cylinder pistons and plural systems of heads having a drive mechanism for moving the piston holders forward and backward so that the device can hold a plurality of syringes and operates injection or suction in each syringe independently. This device also has a mechanism for prohibiting the backward-moving of the piston holder of a second head when the piston holder of a first head is in a forward-moving state and the piston holder of the second head is in a stopped state. This structure effectively prevents liquid from being undesirably mixed and the injection amount thereof from becoming less accurate.

U.S. Publication No. 2018/055995 A1 discloses a controlled delivery drive mechanism including a drive housing, a piston, and a biasing member initially retained in an energized state and is configured to bear upon an interface surface of the piston. The piston is configured to translate a plunger seal and a barrel. A tether is connected between the piston and the winch drum to restrain the free expansion of the biasing member and the free axial translation of the piston upon which the biasing member bears upon. The drive mechanism may further include a gear assembly and an escapement regulating mechanism configured to control the rotation of the gear assembly to release the tether from the winch drum. The metering of the tether by the escapement regulating mechanism controls the rate or profile of drug delivery to a user.

Further, common pumps for administering medicine, such as, for example, insulin pumps, comprise a plurality of fluid or flow paths in order to administer a basal rate, e.g., a base or background amount of insulin, as well as a bolus rate, e.g., an extra or additional amount of insulin. Fluid or flow paths are generally susceptible to occlusions, leading to limitation of the amount of delivered insulin or even failure to deliver insulin at all. As an example, U.S. Pat. No. 6,283,944 B1 discloses a pump having a bulkhead that is provided with the first and second flow paths from the pump reservoir to a single outlet port. Further, an infusion system delivering drug to the patient at a fixed rate while permitting the patient to introduce a controlled bolus dosage when needed is disclosed. Further, WO 2009/045776 A2 discloses a wearable infusion device comprising a first control movable between a first position and a second position that when in the first position, establishes a first fluid path between a reservoir and a pump and when in the second position, establishes a second fluid path between the pump and an outlet. A second control actuates the pump only when the second fluid path has been established by the first control.

It is therefore desirable to provide methods and devices which address the above mentioned technical challenges. Specifically, a drive system, an insulin pump and a method shall be provided providing a high degree of precision and reliability of delivering insulin, while, still, allowing for a small and compact construction.

### SUMMARY

This problem is addressed by a drive system for driving an insulin pump, an insulin pump for delivering insulin to a user and a method for driving an insulin pump with the features of the independent claims. Advantageous embodiments which might be realized in an isolated fashion or in any arbitrary combinations are listed in the dependent claims.

As used in the following, the terms “have,” “comprise” or “include” or any arbitrary grammatical variations thereof are used in a non-exclusive way. Thus, these terms may both refer to a situation in which, besides the feature introduced by these terms, no further features are present in the entity described in this context and to a situation in which one or more further features are present. As an example, the expressions “A has B,” “A comprises B” and “A includes B” may both refer to a situation in which, besides B, no other element is present in A (i.e., a situation in which A solely and exclusively consists of B) and to a situation in which, besides B, one or more further elements are present in entity A, such as element C, elements C and D or even further elements.

Further, it shall be noted that the terms “at least one,” “one or more” or similar expressions indicating that a feature or element may be present once or more than once typically will be used only once when introducing the respective feature or element. In the following, in most cases, when referring to the respective feature or element, the expressions “at least one” or “one or more” will not be repeated, non-withstanding the fact that the respective feature or element may be present once or more than once. It shall be understood for purposes of this disclosure and appended claims that, regardless of whether the phrases “one or more” or “at least one” precede an element or feature appearing in this disclosure or claims, such element or feature shall not receive a singular interpretation unless it is made explicit herein. By way of non-limiting example, the terms “gear,” “ratchet” and “motor,” to name just a few, should be interpreted wherever they appear in this disclosure and claims to mean “at least one” or “one or more” regardless of whether they are introduced with the expressions “at least one” or “one or more.” All other terms used herein should be similarly interpreted unless it is made explicit that a singular interpretation is intended.

Further, as used in the following, the terms “preferably,” “more preferably,” “particularly,” “more particularly,” “specifically,” “more specifically” or similar terms are used in conjunction with optional features, without restricting alternative possibilities. Thus, features introduced by these terms are optional features and are not intended to restrict the scope of the claims in any way. The invention may, as the skilled person will recognize, be performed by using alternative features. Similarly, features introduced by “in an embodiment of the invention” or similar expressions are intended to be optional features, without any restriction regarding alternative embodiments of the invention, without any restrictions regarding the scope of the invention and without any restriction regarding the possibility of combining the features introduced in such way with other optional or non-optional features of the invention.

In a first aspect of this disclosure, a drive system for driving an insulin pump is disclosed. The term “drive system” as used herein is a broad term and is to be given its ordinary and customary meaning to a person of ordinary skill in the art and is not to be limited to a special or customized meaning. The term specifically may refer, without limitation, to an arbitrary system configured to impart a forward motion by exerting a force. In particular, the drive system may set and/or keep a flow of insulin in motion. The drive system may specifically be configured for driving the insulin pump. The term “insulin pump” as used herein is a broad term and is to be given its ordinary and customary meaning to a person of ordinary skill in the art and is not to be limited to a special or customized meaning. The term specifically may refer, without limitation, to a device for administering insulin from at least one insulin reservoir to a user.

The drive system comprises:
a motor configured for rotating at a predetermined revolution speed;
a gear box for converting a rotation of the motor into a continuous linear motion of a piston, wherein the continuous linear motion of the piston determines a basal rate of insulin delivery; and
a mechanical displacement unit configured for superposing the continuous linear motion of the piston by a mechanical displacement of the piston independent of the basal rate.

The term “motor” as used herein is a broad term and is to be given its ordinary and customary meaning to a person of ordinary skill in the art and is not to be limited to a special or customary meaning. The term specifically may refer, without limitation, to an arbitrary engine, machine or device configured for transforming energy into kinetic energy or into a motion, such as for transforming mechanical energy, electrical energy or chemical energy into kinetic energy or into a motion of a device. Thus, the motor may be or may comprise a mechanical motor or an electromechanical motor. Particularly, the motor may transform the energy into a rotation or rotational movement of at least part of the motor itself. Preferably, the motor may be configured for transforming energy from an energy source, such as from an external energy source, into the rotational movement. In particular, the motor may, for example, be an electrically or physically powered motor, such as, for example, an electric motor, a pneumatic motor, a hydraulic motor, a clockwork motor or the like. The rotation, specifically the revolution speed of the rotation of the motor may, for example, be predetermined by the motor, e.g., by the build or construction of the motor itself, or by the amount or level of energy supplied to the motor.

The term “energy” as used herein is a broad term and is to be given its ordinary and customary meaning to a person of ordinary skill in the art and is not to be limited to a special or customary meaning. The term specifically may refer, without limitation, to an arbitrary form of power provided to the motor. In particular, the energy may be an electrical energy, a kinetic energy, a potential energy or the like. As an example, the energy may be provided in the form of an electric current, one or more elastic elements, e.g., springs or elastic bands, a compressed gas or in form of a pressurized fluid.

The rotation of the motor is converted into the continuous linear motion of the piston by the gear box. The term “gear box” as used herein is a broad term and is to be given its ordinary and customary meaning to a person of ordinary skill in the art and is not to be limited to a special or customary meaning. The term specifically may refer, without limitation, to a mechanical system or device configured for modifying or converting a speed, a direction and/or a force of a motion, such as the speed, torque, direction and/or force of a linear or rotational movement. In particular, the gear box may convert the speed, torque, direction and/or force of a the movement by using a variety of transmission elements, such as, for example, gears, wheels, levers, belts, toothed racks or any other elements configured for transmitting movement. Specifically, the gear box may comprise an arbitrary combination of such transmission elements in order to convert the movement according to the requirements. Particularly, the gear box is configured for converting the rotation of the motor into the continuous linear motion of the piston. Specifically, the gear box may be configured for acting on the piston, e.g., on the piston of the insulin pump.

The term “continuous linear motion” as used herein is a broad term and is to be given its ordinary and customary meaning to a person of ordinary skill in the art and is not to be limited to a special or customized meaning. The term specifically may refer, without limitation, to an uninterrupted movement in an arbitrary direction along an axis, wherein the axis specifically may differ from a linear axis by no more than 20%, preferably by no more than 10%, more preferably by no more than 5%. The uninterrupted movement, as an example, may take place at a constant speed, such as at a speed which deviates from a mean value by no more that 20%, preferably by no more than 10%, more preferably by no more than 5%. It shall be noted, however, that motions with varying speed are also possible.

The term “piston” as used herein is a broad term and is to be given its ordinary and customary meaning to a person of ordinary skill in the art and is not to be limited to a special or customized meaning. The term specifically may refer, without limitation, to an arbitrary movable element whose movement effectuates or produces a movement of a fluid, specifically insulin, wherein the fluid may be in direct or in indirect contact with the piston. The piston, as an example, may be or may comprise a plunger. In particular, the piston may, for example, be or may comprise a moveable wall or surface, such as a front surface or front wall of a plunger, specifically a moveable wall of a containment, such as, for example, a cartridge or case. The piston may preferably be configured for displacing a content of the containment, such as, for example, insulin, preferably if the piston is moved. In particular, the piston may be movable within a cartridge or case configured for holding the fluid, e.g., insulin. The movement of the piston may specifically extrude the fluid, e.g., insulin, from the cartridge or case. As an example, the movement of the piston may lead to an extrusion of the insulin from an insulin reservoir. Preferably, the piston may be or may comprise at least one material which provides a moveable sealing between the piston and the cartridge, specifically between the piston and a wall of the cartridge, such as, for example, a flexible material. As an example, the piston may be or may comprise at least one elastomeric material, such as, for example, any type of rubber or thermoplastic elastomer. However, different types of piston material may exist. The piston may in particular be configured for performing the continuous linear motion, wherein the continuous linear motion of the piston determines the basal rate of insulin delivery.

The term “basal rate” as used herein is a broad term and is to be given its ordinary and customary meaning to a person of ordinary skill in the art and is not to be limited to a special or customized meaning. The term specifically may refer, without limitation, to a fundamental quantity or volume or to a fundamental or predetermined volume flow rate of fluid, specifically insulin. Specifically, the basal rate of insulin may be the minimum amount or volume flow rate of insulin essential or fundamental for controlling cellular glucose and amino acid uptake in a human or animal body. As an example, the basal rate of insulin may regulate a blood glucose level caused by a glucose output of a liver in a human or animal body.

Further, the mechanical displacement unit is configured for superposing the continuous linear motion of the piston by the mechanical displacement of the piston independent of the basal rate. Specifically, the continuous linear motion of the piston determining the basal rate of insulin delivery may be superposed by the mechanical displacement of the piston. The term “mechanical displacement unit” as used herein is a broad term and is to be given its ordinary and customary meaning to a person of ordinary skill in the art and is not to be limited to a special or customized meaning. The term specifically may refer, without limitation, to an arbitrary device configured for directly or indirectly performing or operating a mechanical displacement of an object or element. The term “mechanical displacement” as used herein is a broad term and is to be given its ordinary and customary meaning to a person of ordinary skill in the art and is not to be limited to a special or customized meaning. The term specifically may refer, without limitation, to physically changing or adjusting a position of an object or element. In particular, the mechanical displacement of the piston independent of the basal rate may be performed by the mechanical displacement unit. Specifically, the continuous linear motion of the piston determining the basal rate of insulin delivery may be superposed by the mechanical displacement. Preferably, the movement of the piston in order to extrude the basal rate may be performed manually. Additionally or alternatively the movement of the piston for extruding the basal rate may be performed hydraulically, such as, for example, by using a hydraulic fluid to exert pressure onto the piston.

The term “superposed” as used herein is a broad term and is to be given its ordinary and customary meaning to a person of ordinary skill in the art and is not to be limited to a special or customized meaning. The term specifically may refer, without limitation, to the process of adding or layering, specifically adding one motion to another or overlaying a first motion with at least one second motion. In particular, if the continuous linear motion of the piston is superposed by the mechanical displacement of the piston, the piston may perform the mechanical displacement additionally to the continuous linear motion. In particular, the piston continues its continuous linear motion while additionally performing the mechanical displacement.

Further, the mechanical displacement of the piston independent of the basal rate may define a bolus of insulin delivery. In particular, the mechanical displacement of the piston may specifically extrude the bolus of insulin from the cartridge or case. As an example, the mechanical displacement of the piston may lead to an extrusion of the bolus of insulin from an insulin reservoir. The term “bolus” as used herein is a broad term and is to be given its ordinary and customary meaning to a person of ordinary skill in the art and is not to be limited to a special or customized meaning. The term specifically may refer, without limitation, to a dosage or discrete amount of fluid. Specifically, the bolus of insulin may be the amount of insulin essential for controlling cellular glucose and amino acid uptake in the human or animal body after a meal. As an example, the bolus of insulin may regulate a blood glucose level caused by the intake of a meal into the human or animal body.

The drive system may further comprise at least one energy source. The energy source specifically may be designed for storing and releasing an amount of energy, such as mechanical or electrical energy. The energy source may specifically be configured for providing energy to the motor. As indicated above, the energy may be an electrical energy, a kinetic energy, a potential energy or the like. Thus, the energy source may particularly be an arbitrary device configured to provide energy in the form of an electric current, one or more elastic elements, e.g., springs or elastic bands, a compressed gas or in form of a pressurized fluid, specifically to the motor. Additionally, the energy source may be configured for storing a predefined amount of said energy. Preferably, the at least one energy source may comprise at least one electrical energy source. Specifically, the electrical energy source comprised by the energy source may be or may comprise at least one of a battery or an accumulator.

As indicated above, the motor may, for example, be an electrically or physically powered motor. Specifically, the motor may comprise at least one motor selected from the group consisting of: an electrical motor, a clockwork, preferably a spring driven clockwork.

Further, the piston may be mechanically coupled to a threaded rod. The term “threaded rod” as used herein is a broad term and is to be given its ordinary and customary meaning to a person of ordinary skill in the art and is not to be limited to a special or customized meaning. The term specifically may refer, without limitation, to an arbitrary elongated element fully or partially spirally grooved. In particular, the threaded rod may be a shaft or stem having at least one spiral groove around an axis of the threaded rod. Specifically, the threaded rod may be a long screwlike element having two ends. Preferably, the threaded rod may comprise a solid material. For example, the threaded rod may be made of a plastic material, a metal material or metal alloy material, or a combination thereof. In particular, the piston may be mechanically coupled to the at least one end of the threaded rod. As an example, the piston may be mechanically coupled to the at least one end of the threaded rod by a plain contact, for example, a physical contact allowing to push the piston forward. Additionally or alternatively, the piston may be mechanically coupled to the threaded rod by a push-pull coupling, such as a push-pull coupling providing a fixed physical contact, preferably a push-pull coupling capable of preventing the piston from losing contact to the rod. As an example, the piston may be screwed onto the threaded rod, specifically one end of the threaded rod may be inserted into the piston by rotating the threaded rod relative to the piston. Other options or mechanisms for mechanically coupling the piston to the threaded rod are feasible, such as, for example, a bayonet-coupling or a plug connection. In particular, the movement of the piston may be controlled by an axial movement of the threaded rod.

The mechanical coupling of the piston to the threaded rod may specifically be configured to transmit motion of the one onto the other. Specifically, the piston and the threaded rod may be mechanically coupled such that a motion of the threaded rod leads to a motion of the piston or vice versa. In particular, a continuous linear motion of the piston may equal a continuous linear motion of the threaded rod. Specifically, a motion of the threaded rod may be directly transferred onto the piston. Thus, a movement of the threaded rod, e.g., a continuous linear motion of the threaded rod, may lead to the piston performing the same movement, e.g., the same continuous linear motion, as the threaded rod, or vice versa. In particular, the gear box may, for example, convert the rotation of the motor into the continuous linear motion of the piston via the threaded rod.

Additionally or alternatively, the mechanical coupling of the piston and the threaded rod may be configured such that a mechanical displacement of the piston may equal a mechanical displacement of the threaded rod. Specifically, a mechanical displacement of the threaded rod may lead to a mechanical displacement of the piston. In particular, the mechanical displacement unit may, for example, be configured for acting on the piston by superposing the continuous linear motion of the piston by a mechanical displacement of the threaded rod.

In particular, the threaded rod may preferably have a thread, such as a metric thread or fine thread. In particular, the thread may have a gradient or thread pitch ranging from 0.1 mm to 1.5 mm, preferably ranging from 0.3 mm to 1 mm, more preferably from 0.5 mm to 0.8 mm. As an example, the pitch of the threaded rod may correspond to a cross-sectional area of the piston. Specifically, the pitch of the threaded rod may correspond to the cross-sectional area of the piston, such that by moving the threaded rod by one turn of the thread, a specific amount of insulin, such as the amount of insulin representing the bolus, may be displaced, e.g., extruded, from the cartridge. As indicated above, the movement of the piston may depend on the movement of the threaded rod. As an example, a movement of the piston of 0.7 mm along the axis of the threaded rod may, for example, lead to an extrusion or administration of 5 IU.

Further, the motor may be coupled to the threaded rod via at least one gear. In particular, the gear may be comprised by the gear box. Specifically, the rotation of the motor may be transmitted onto the threaded rod via the gear. The gear may further be mounted concentrically about the threaded rod. Specifically, the gear may be mounted in a concentric fashion about the axis of the threaded rod. The term “gear” as used herein is a broad term and is to be given its ordinary and customary meaning to a person of ordinary skill in the art and is not to be limited to a special or customized meaning. The term specifically may refer, without limitation, to an arbitrary object configured for transmitting motion by rotating about an axis. As an example, the gear may be a toothed wheel, a worm, a friction wheel or the like. In particular, the gear may be configured for transmitting motion using various transfer mechanisms, such as traction or positive locking, for example, by interlocking or meshing teeth.

The threaded rod may particularly be secured against rotation about its axis. Preferably, the threaded rod may be secured against rotating about its axis by at least one of a bolt or a toggle. As an example, the bolt or toggle may be arranged such as to prevent the rotation of the threaded rod. For example, the toggle or bolt may be mounted transversely to the axis of the threaded rod.

Further, the gear may be mounted to the threaded rod by a coupling. In particular, the coupling may have at least two engagement elements capable of engaging with the threaded rod at at least two axially displaced engagement positions.

In particular, the engagement elements may each comprise at least one ratchet. Specifically, the ratchet may be configured for imparting, governing and/or preventing a movement of the engagement element in at least one direction. The engagement elements, with their respective ratchets, specifically may form a double-ratchet arrangement. The term “double ratchet arrangement” as used herein is a broad term and is to be given its ordinary and customary meaning to a person of ordinary skill in the art and is not to be limited to a special or customized meaning. The term specifically may refer, without limitation, to an arbitrary system of two or more ratchets configured for engaging with one other element, such as, for example, with the threaded rod, wherein at least one of the at least two ratchets is always engaged with the other element, e.g., with the threaded rod.

Further, the engagement elements may each comprise a rigid base surrounding the threaded rod. Specifically, the rigid base may, for example, be ring-shaped and may be arranged around the threaded rod, such as to surround the threaded rod. Additionally, the engagement elements may each comprise ratchet arms extending in an axial direction from the rigid base. The ratchet arms may specifically be configured to engage with at least one thread of the threaded rod. Preferably, the ratchet arms configured for engaging with the thread may impart, govern and/or prevent the movement of the engagement elements. Specifically, the ratchet arms may extend from the rigid base in a direction towards the piston.

Further, the engagement elements may be mounted on the threaded rod in a fashion that the engagement elements may be shiftable with respect to one another. In particular, the engagement elements may be movable along the axis of the threaded rod with respect to one another. Specifically, the engagement elements may be shiftable, such that, for example, the distance between the at least two axially displaced engagement positions of the engagement elements may be varied or changed.

Preferably, the engagement elements may be connected via at least two bearing rods. In particular, at least one of the engagement elements may be mounted on the bearing rods in an axially shiftable fashion. Preferably, the at least two bearing rods may connect the engagement elements in such fashion, that the engagement elements may be movable along the axis of the threaded rod with respect to each other. As an example, the movement of the engagement elements may be guided along the bearing rods, specifically along the axis of the threaded rod.

Further, the engagement elements may be connected via at least one axially acting spring element. Specifically, the engagement elements may be connected via the at least one axially acting spring element, such as, for example, an axially extending spring element. In particular, the spring element may connect the engagement elements. The spring element may specifically be arranged, such that the spring element surrounds at least one of the bearing rods. Preferably, the engagement elements may be connected via two axially acting spring elements, wherein each spring element may surround one of the two bearing rods.

The engagement elements may be connected in a rotationally fixed fashion. In particular, the engagement elements may be connected, such that a rotational movement of one of the engagement elements, specifically a rotation around the axis of the threaded rod, leads to a similar rotational movement of the other engagement element. Preferably, one engagement element may not be able to rotate relative to the other engagement element. As an example, the engagement elements may be connected in a rotationally fixed fashion via the at least two bearing rods.

Further, the coupling may comprise at least three coupling states. In particular, at least three coupling states may be adoptable by the coupling. The three coupling states may particularly be:
a first state, wherein in the first state both engagement elements may engage with the threaded rod. Further, the threaded rod may be driven in an axial direction specifically by a rotation of the gear about an axis of the threaded rod. In particular, in the first state, the engagement elements may be located at a fixed spatial separation from each other;
a second state, wherein in the second state a first engagement of the engagement elements may engage with the threaded rod and a second engagement of the engagement elements may disengage with the threaded rod. Specifically, the first engagement element may push the threaded rod through the second engagement element in a first axial direction towards the piston; and
a third state, wherein the first engagement element may disengage with the threaded rod and the second engagement element may engage with the threaded rod. Particularly, in the third state the first engagement element may be pushed back in a second axial direction opposing the first axial direction.

As an example, in the first state, the engagement elements may have a fixed axial separation. Specifically, a fixed axial separation, such as a predefined distance, may exist between the engagement elements in the first state, particularly in the first state adoptable by the coupling.

In particular, the mechanical displacement of the piston independent of the basal rate may be performed in the second state. Specifically, as indicated above, the piston may be mechanically coupled to the threaded rod. Thus, the threaded rod being pushed through the second engagement element may lead to a movement of the piston, preferably to the mechanical displacement of the piston.

Further, the gear may be driven by the motor via a drive. The term “drive” as used herein is a broad term and is to be given its ordinary and customary meaning to a person of ordinary skill in the art and is not to be limited to a special or customized meaning. The term specifically may refer, without limitation, to an arbitrary device for transmitting and/or converting movement or motion. In particular, the drive may be a transmission or gearing mechanism. As an example, the drive may specifically be configured for transmitting and/or converting motion, such as, for example, the rotation of the motor, onto the gear. Preferably, the drive may have a transmission or gear ratio 1. In particular, the drive may convert or transform the rotation of the motor onto the gear, such that the rotation speed of the gear may differ from the rotation speed of the motor. Preferably, the drive may transmit the rotation of the motor onto the gear, such that the gear rotation may be slower than the rotation of the motor. In particular, the motor may, for example, be a motor, specifically a type of motor, with permanent magnets for an excitation field. As an example, this type of motor may provide a speed, specifically a speed of the rotational movement of an output shaft, wherein the speed may depend on an applied voltage and wherein the speed may further depend on a load, specifically on a load at the output shaft of the motor. As an example, an increase of the load may result in a decrease of the speed, such that as an example more load may result in lower speed. In particular, a change in speed, preferably a change of the rotation speed, may result from a change of the load. As an example, depending on the motor layout, the change of speed as an answer to a defined change of load may be large or small. Preferably, as an example the change of speed as an answer to a change of the load may be small. As an example, a motor may exist, which may inherently provide this characteristic, for example, by or as a result of its internal dimensions. As an example, the internal dimensions of the motor may, for example, be or may comprise a strength of the magnets, a cross-section of a working airgap, a number of windings of one or more coils, or the like.

As an example, the gearbox ratio, specifically a transmitting ratio of the drive and the gear, may specifically be chosen in order to achieve, for example, two effects. In particular, as a first effect, the speed of the motor may be adapted such that the speed of the piston, specifically the speed of the piston driven by the threaded rod, may lead to the extrusion of the basal rate of insulin. Thus, as an example, first the speed of the motor may be reduced to an appropriate speed of the thread in order to, for example, provide the desired displacement of insulin over time. Further, in particular as a second effect, the gear box, specifically the drive and the gear, may, for example, have a ratio, for example, a gear ratio, that may provide such a leverage of torque from the motor to the thread that, as an example, the motor may run more or less on idle. This may, for example, provide an inherent speed regulation, specifically the speed of the piston may be regulated inherently. In particular, the inherent speed regulation may, for example, be provided as long as a voltage for driving the motor may remain substantially constant. speed ratio of the rotation of the gear and the rotation of the motor may, for example, be in the As an example, the speed ratio of the rotation of the gear and the rotation of the motor may, for example, be in the range from 1·10⁻⁴ to 1·10⁻⁸, preferably in the range from 1·10⁻⁵ to 1·10⁻⁷, more preferably in the range from 5·10⁻⁵ to 5·10⁻⁶. Further, the gear, via the coupling, may drive the threaded rod. Specifically, the gear may drive the threaded rod via the coupling having at least two engagement elements preferably capable of engaging with the threaded rod at at least two axially displaced engagement positions.

Further, the gear and at least one of the engagement elements may be mounted together. In particular, the gear may be mounted together with a bushing and the at least one of the engagement elements to form a unit. As an example, the gear may be clamped between the at least one of the engagement elements and the bushing. The term “bushing” as used herein is a broad term and is to be given its ordinary and customary meaning to a person of ordinary skill in the art and is not to be limited to a special or customized meaning. The term specifically may refer, without limitation, to an arbitrary cylindrical object adapted to a lateral surface of the gear. As an example, the bushing may be a cylindrical lining adapted to the lateral surface of the gear. In particular, the bushing may, for example, be configured for mechanically stabilizing or protecting the gear. As an example, the bushing may be configured for resisting abrasion, force absorption or the like. In particular, the bushing may be part of the unit, wherein the unit may specifically be axially fixed, such that the unit may, for example, be caught in an axial position. Additionally, as an example, the unit may be configured to perform a rotational movement. Specifically, as an example, the unit may further rotate freely. In particular, the unit, specifically the gear mounted together with the engagement element, may, for example, move in a combination of radial and axial bearings, specifically to exert an axial force onto the threaded rod.

The drive system may further comprise at least one spring abutment concentrically mounted about the threaded rod. Preferably, the at least one spring abutment may be configured for limiting a movement of the coupling in a direction away from the piston. The term “spring abutment” as used herein is a broad term and is to be given its ordinary and customary meaning to a person of ordinary skill in the art and is not to be limited to a special or customized meaning. The term specifically may refer, without limitation, to a mechanical stop or catch. Specifically, the spring abutment may be configured for limiting a movement of the coupling, preferably by limiting a movement of the axially acting spring element.

Further, the mechanical displacement unit may comprise at least one displacement lever configured for one or both of exerting an axial pressure onto the piston or axially displacing the piston. The term “displacement lever” as used herein is a broad term and is to be given its ordinary and customary meaning to a person of ordinary skill in the art and is not to be limited to a special or customized meaning. The term specifically may refer, without limitation, to an arbitrary device formed to exert a mechanical displacement when being activated. In particular, the displacement lever may, for example, have a handle shape. Specifically, the displacement lever may be configured for exerting the axial pressure onto the piston via the threaded rod and/or for axially displacing the piston via the threaded rod. In particular, the displacement lever may be configured for exerting the axial pressure onto the threaded rod via at least one of the engagement elements and via the threaded rod onto the piston. As an example, the displacement lever, specifically when activated, may trigger the coupling to switch from the first coupling state to the second coupling state to the third coupling state and back to the first coupling state, by exerting an axial pressure onto the first engagement element.

In particular, the drive may be or may comprise an arbitrary arrangement or combination of any number of gear elements. Specifically, the drive may, for example, comprise one or more of a toothed wheel, a worm gear, a friction wheel, a belt drive, a chain drive, or the like. Other gear elements and/or combinations thereof may be feasible.

The drive may further comprise at least one gearwheel for adjusting and/or defining a transmission ratio between the motor and the gear. Preferably, the drive may comprise more than one gearwheel, such as, for example, a combination of several gear elements, for defining the transmission ratio between the motor and the gear. The term “gearwheel” as used herein is a broad term and is to be given its ordinary and customary meaning to a person of ordinary skill in the art and is not to be limited to a special or customized meaning. The term specifically may refer, without limitation, to an arbitrary wheel shaped object configured for transmitting motion by rotating about an axis. As an example, the gearwheel may be a toothed wheel, a worm gear, a friction wheel, or the like.

Further, the drive may comprise at least one belt for transmitting the rotation of the motor onto the gear. In particular, the belt may, for example, be a toothed belt or a friction belt configured for transmitting the rotation of the motor onto the gear via positive locking, e.g., by meshing teeth, traction or the like.

In particular, the drive may comprise at least one endless screw configured for transmitting and/or converting the rotation of the at least one gearwheel onto the gear in an orthogonal fashion. Preferably the endless screw may, for example, be a worm, specifically a worm in mesh with a worm shaft, wherein the worm shaft may also be comprised by the drive.

Specifically, the belt may interact with a first gearwheel thereby transmitting the rotation of the motor onto the first gearwheel. Further, the first gearwheel may interact with a second gearwheel. Thus, preferably the rotation of the first gearwheel may be transmitted onto the second gearwheel. The second gearwheel may further interact with a third gearwheel. Preferably, the interaction between the second gearwheel and the third gearwheel may lead to a transmission of the rotation of the second gearwheel onto the third gearwheel. Further, the third gearwheel may interact with a fourth gearwheel. Preferably, the interaction between the third gearwheel and the fourth gearwheel may lead to a transmission of the rotation of the third gearwheel onto the fourth gearwheel. Further, the fourth gearwheel may interact with a fifth gearwheel. Preferably, the interaction between the fourth gearwheel and the fifth gearwheel may lead to a transmission of the rotation of the fourth gearwheel onto the fifth gearwheel. The fifth gearwheel may preferably be connected to the endless screw, wherein the endless screw may interact with the gear. Preferably, a rotation axis of the endless screw may be arranged orthogonally to a rotation axis of the gear.

The drive system may further comprise a mechanical occlusion detection system. The term “mechanical occlusion detection system” as used herein is a broad term and is to be given its ordinary and customary meaning to a person of ordinary skill in the art and is not to be limited to a special or customized meaning. The term specifically may refer, without limitation, to an arbitrary mechanical system configured for providing information on a state or condition of insulin flow. In particular, the mechanical occlusion detection system may be configured for detecting a blockage or occlusion of insulin delivery. Preferably the mechanical occlusion detection system may be configured for providing information, such as, for example, visual information, on the blockage or occlusion to the user, such as, for example, through a viewing window. As an example, the mechanical occlusion detection system may be part of the gear box, such as a gearwheel being visually detectable, such as, for example, being of contrasting color or having at least one mark. Thus, preferably, the visually detectable gearwheel may be part of the gear box or the drive, and may be visible for the user. In particular, a motionless gearwheel may indicate an occurrence or existence of the blockage or occlusion. Additionally or alternatively, the mechanical occlusion detection system may be fully or partially arranged in a flow path, particularly in a flow path of the insulin, such as, for example, inside a tube.

As an example, the mechanical occlusion detection system may comprise at least one of an impeller or an Archimedes' screw. Specifically, the impeller or the Archimedes' screw may be arranged inside the flow path of the insulin. Via a shaft, the impeller or the Archimedes' screw may drive a shaft. The shaft may further be connected to a wheel, wherein a rotation of the wheel may be visually detectable by the user, e.g., by a marking or color of the wheel. In particular, a surface wetted by the insulin may, for example, be greater when using the Archimedes' screw than when using the impeller, thus as an example, the Archimedes' screw may, for example, allow for a visual detection of smaller flow rates of insulin than the impeller.

Additionally or alternatively, the mechanical occlusion detection system may, for example, comprise an elastic element, preferably an elastically deformable element, such as, for example, an elastic membrane. Specifically, the elastic element may bulk when applied with a pressure or force and may be arranged such as to being visually detectable by the user. Preferably, the elastic element may further be arranged such as to be in contact with the insulin, e.g., the insulin inside the flow path. In case of an occurring occlusion or blockage, the pressure, in particular the fluidic pressure of the insulin, inside the flow path may increase. Thus, the elastic membrane being in contact with the insulin may bulk in the case of an occurring occlusion. In order to increase visibility of the membrane by the user, the membrane may, for example, be configured for changing its color when bulking and/or a magnifying glass may be arranged such as to magnify the size of the membrane, thus increasing visibility of the membrane.

Additionally or alternatively, a float or a suspended body may be arranged in a pressure tube of the flow path. As an example, the pressure tube may be arranged orthogonal or parallel to the flow path of the insulin. In case of an occurring occlusion or blockage, the pressure, in particular the fluidic pressure of the insulin, inside the flow path may increase. Thus, the float or suspended body may be lifted or elevated by the fluidic pressure of the insulin. Preferably the lifted or elevated position of the float or suspended body may be visible to the user. Thus, an occurring occlusion may be visible to the user by checking the position of the float. Preferably, the float or suspended body may be connected to the pressure tube by a spring configured for restoring a position of the float when insulin is flowing, e.g., no blockage of insulin exists.

Additionally or alternatively, an object may be arranged in the flow path, preferably on an inclined plane in the flow path. The velocity of the flowing insulin may keep the object at a suspended position on the inclined plane. In case of an occurring occlusion or blockage of the insulin flow, the object may sink on the inclined plane. Preferably, the position of the object on the inclined plane may be visible to the user, providing information on the state of insulin flow. Preferably, the object may be connected to a low end of the inclined plane by a spring configured for ensuring the sinking of the body when a blockage exists, e.g., insulin is not flowing.

In a further aspect, an insulin pump for delivering insulin to a user is disclosed. The insulin pump comprises at least one insulin reservoir. Further, the insulin pump comprises at least one drive system, specifically, the drive system as indicated above or as further indicated below. The term “insulin reservoir” as used herein is a broad term and is to be given its ordinary and customary meaning to a person of ordinary skill in the art and is not to be limited to a special or customized meaning. The term specifically may refer, without limitation, to a hollow element or container which may fully or partially be filled with insulin. Specifically, the insulin reservoir may comprise at least one cartridge or vial which specifically may removably be placed within the insulin pump.

Further, the insulin pump may comprise a housing. As used herein, the term “housing” is a broad term and is to be given its ordinary and customary meaning to a person of ordinary skill in the art and is not to be limited to a special or customized meaning. The term specifically may refer, without limitation, to a basically arbitrary element which is configured for fully or partially enclosing one or more components and for providing protection for these one or more components, such as against mechanical influence and/or humidity. The housing specifically may be or may comprise a rigid housing, such as a rigid housing made of one or more plastic material, a metallic material or combinations thereof. Specifically, the drive system may fully or partially be arranged in the housing. Additionally the insulin reservoir may be fully or partially arranged in the housing. In particular, the housing may, for example, have at least one viewing window configured for enabling the user to visually check the information on the state or condition of insulin flow provided by the mechanical occlusion detection system.

The insulin pump may further comprise a starting element. The starting element may specifically be configured for starting delivering insulin using the insulin pump.

In a further aspect, a method for driving an insulin pump is disclosed. The method comprises the steps disclosed in the following. The steps may specifically be performed in the given order. Still, a different order is possible. The method may comprise additional steps which are not mentioned. It is further possible to perform one or more or all of the method steps repeatedly. Further, two or more of the method steps may be performed simultaneously or in a timely overlapping fashion.

The method comprises the following steps:
a) rotating a motor at a predetermined revolution speed;
b) converting a rotation of the motor into a continuous linear motion of a piston by using a gear box, wherein the continuous linear motion of the piston determines a basal rate of insulin delivery; and
c) superposing the continuous linear motion of the piston by a mechanical displacement of the piston independent of the basal rate, by using a mechanical displacement unit.

For possible definitions and options, reference may be made to the description of the drive system or the insulin pump given above. The method may specifically be configured for driving the insulin pump described above.

The method may further comprise delivering a bolus of insulin, the bolus of insulin being defined by the mechanical displacement of the piston independent of the basal rate.

In particular, the method may further comprise providing energy, specifically electrical energy, to the motor by using at least one energy source. In particular, the method may comprise providing electrical energy to the motor by using at least one of a battery or an accumulator. The motor may specifically comprise at least one motor selected from the group consisting of an electrical motor, a clockwork or the like. Specifically, the clockwork may preferably be a spring driven clockwork.

Method step b) specifically may comprise mechanically coupling the piston to a threaded rod. Further, method step b) may comprise coupling the motor to the threaded rod. Specifically, coupling the motor to the threaded rod via at least one gear. Additionally, method step b) may further comprise mounting the gear concentrically about the threaded rod. Further, method step b) may comprise securing the threaded rod against rotation about its axis, preferably by using at least one of a bolt or a toggle.

In particular, method step b) may further comprise mounting the gear to the threaded rod by a coupling, the coupling having at least two engagement elements capable of engaging with the threaded rod at at least two axially displaced engagement positions. Specifically, the engagement elements may each comprise at least one ratchet. In particular, the engagement elements may each comprise a rigid base surrounding the threaded rod and ratchet arms extending in an axial direction from the rigid base. Preferably, method step b) may further comprise engaging the ratchet arms with at least one thread of the threaded rod. Wherein, in particular, the ratchet arms may extend from the rigid base in a direction towards the piston.

The engagement elements may preferably be mounted on the threaded rod in a fashion that the engagement elements are shiftable with respect to one another. In particular, the engagement elements may be connected via at least two bearing rods, wherein preferably at least one of the engagement elements may be mounted on the bearing rods in an axially shiftable fashion.

In particular, the engagement elements may be connected via at least one axially acting spring element. Further, the engagement elements may be connected in a rotationally fixed fashion.

The coupling may specifically comprise at least three coupling states. Preferably, three coupling states may be adoptable by the coupling. The three coupling states may specifically be:
a first state, wherein in the first state both engagement elements may engage with the threaded rod, wherein the threaded rod may be driven in an axial direction by a rotation of the gear about an axis of the threaded rod, wherein in the first state, the engagement elements may be located at a fixed spatial separation from each other;
a second state, wherein in the second state a first engagement of the engagement elements may engage with the threaded rod and a second engagement of the engagement elements may disengage with the threaded rod, wherein the first engagement element may push the threaded rod in an axial direction through the second engagement element in a first axial direction towards the piston; and
a third state, wherein the first engagement element may disengage with the threaded rod and the second engagement element may engage with the threaded rod, wherein in the third state the first engagement element may be pushed back in a second axial direction opposing the first axial direction.

Particularly, in the first state the engagement elements may preferably have a fixed axial separation. Specifically, a fixed axial separation, such as a predefined distance, may exist between the engagement elements in the first state, particularly in the first state adoptable by the coupling.

Method step c) may particularly comprise the second state. In particular, the first engagement of the engagement elements engaging with the threaded rod and the second engagement of the engagement elements disengaging with the threaded rod may be performed in method step c). Further, method step c) may comprise pushing the threaded rod in an axial direction through the second engagement element in a first axial direction towards the piston by using the first engagement element.

Method step b) may specifically comprise driving the gear by the motor via a drive. In particular, method step b) further may comprise driving the threaded rod by the gear via the coupling. Method step b) may particularly comprise clamping the gear between at least one of the engagement elements and a bushing. Additionally or alternatively, method step b) specifically may comprise mounting at least one spring abutment concentrically about the threaded rod and via the spring abutment limiting a movement of the coupling in a direction away from the piston.

In particular, method step c) may comprise one or both of exerting an axial pressure onto the piston or axially displacing the piston, specifically via the threaded rod, by a displacement lever, wherein the displacement lever is comprised by the mechanical displacement unit.

Specifically, method step b) may comprise adjusting a transmission ratio between the motor and the gear by using at least one gearwheel, preferably more than one gearwheel, comprised by the drive. In particular, method step b) may further comprise transmitting the rotation of the motor onto the gear by using at least one belt, for example, a toothed belt, comprised by the drive. Further, method step b) may comprise converting the rotation of the at least one gearwheel onto the gear in an orthogonal fashion by using at least one endless screw, for example, using a worm.

Specifically, method step b) may further comprise various substeps. In particular, method step b) may comprise the following substeps:
b1) transmitting the rotation of the motor onto the at least one gearwheel;
b2) transmitting the rotation of the gearwheel onto the endless screw; and
b3) transmitting the rotation of the endless screw onto the gear, such that an axis of rotation of the endless screw is arranged orthogonally to an axis of rotation of the gear.

In particular, substep b1) may comprise transmitting the rotation of the motor onto a first gearwheel of a chain of gearwheels comprising at least two gearwheels, preferably three gearwheels, more preferably four gearwheels.

Further, method step b2) may preferably comprise transmitting the rotation of the last gearwheel of the chain of gearwheels onto the endless screw, for example, onto the worm.

Preferably, the method may further comprise detecting an occlusion by using a mechanical occlusion detection system. As an example an unwanted occlusion or blockage within the fluid path of the insulin pump, may be detected.

As an example, the basal rate delivered by using the insulin pump, may preferably be predefined. Alternatively, the basal rate may, for example, be set to a desired rate by the user. In particular, the user may, for example, set the basal rate by operating a dial, a switch, button or a slide. The basal rate may also be set or adjustable by using at least one electronic interface or user operating interface.

The insulin reservoir may preferably be prefilled. Alternatively, the user may be required to fill the insulin reservoir. Specifically, in case the insulin reservoir is not prefilled, the user may fill the insulin reservoir via a syringe, preferably by injecting the insulin into the insulin reservoir, e.g., into a cartridge. For example, the insulin may be injected into the cartridge through a septum, specifically a septum configured for sealing the interior of the cartridge from the outside of the cartridge. Excessive air inside the insulin reservoir may leave the insulin reservoir through an opening sealed against liquids.

Priming of the insulin pump, specifically displace any existing air in the flow path by insulin, may preferably be performed by, for example, activating the mechanical displacement lever for a plurality of times. Specifically, the mechanical displacement lever may exert axial pressure onto the piston, such that, for example, the piston may extrude insulin from the reservoir into the flow path, thereby, for example, displacing any existing air. Alternatively, the priming of the insulin pump may be performed by moving the insulin reservoir further towards the piston, thereby displacing any existing air in the flow path. Additionally, an existing initial breakaway force, specifically an increased friction between the piston and the insulin reservoir may, for example, be overcome by priming the insulin pump, specifically by activating the mechanical displacement lever or by moving the insulin reservoir further towards the piston.

The user may specifically remove an insulation layer, such as an insulating foil or the like, from the power source, such as from the battery or accumulator, in order to start the insulin pump, thereby specifically starting the insulin delivery.

For delivering the bolus, the user may activate the displacement lever. Specifically, activating the displacement lever may deliver a predefined amount of insulin. In particular, the predefined amount of insulin delivered when activating the displacement lever may, for example, depend on the thread, specifically the thread pitch, of the threaded rod.

The occlusion detection system may inform the user of an occurring occlusion or blockage. In particular, the user may be able to visually detect a delivery of insulin, for example, by the information provided by the occlusion detection system. Further, the insulin fill level of the reservoir may also be visually detectable by the user.

After use, the insulin pump may preferably be disposed. In particular, the insulin pump may be disposed after all the insulin from the insulin reservoir may have been delivered to the user.

The proposed devices and method provide a large number of advantages over known methods and devices of similar kind.

In particular, by using the devices and methods as described herein, a drive system, an insulin pump and a method may be provided for reliably and safely delivering insulin. Specifically, the provided method and devices may, for example, forego a need of an electronic control and, as an example, no display may be required. Specifically, the mechanical components may, for example, allow for an inherent security. In particular, the proposed method and devices may, for example, be powered by a battery or an accumulator, specifically providing a specific maximum voltage, thus, as an example, administration a rate of insulin higher than the predefined rate may not be possible. Specifically, the basal rate may be predefined. Further, administration of additional medicine may, for example, be possible, such as, for example, by adding the additional medicine to the reservoir, specifically to the insulin reservoir.

Further, as an example, the simple construction and reduced number of parts used in the proposed method and devices may allow for a more economic production than state of the art methods and devices. In particular, the proposed devices may, for example, be disposable, leading to a variety of advantages of known reusable products. In particular, as an example, material resistance to caustic and abrasive cleaning products may, for example, not be an issue.

Further, various sizes of insulin reservoirs may be usable in the proposed devices, thus as an example allowing for adjusting to individual needs of the user. Specifically, the proposed insulin pump and drive system may, as an example, be able to help users transit from using an insulin pen to using an insulin pump.

Summarizing and without excluding further possible embodiments, the following embodiments may be envisaged:

Embodiment 1: A drive system for an insulin pump comprising:
a motor configured for rotating at a predetermined revolution speed;
a gear box for converting a rotation of the motor into a continuous linear motion of a piston, wherein the continuous linear motion of the piston determines a basal rate of insulin delivery; and
a mechanical displacement unit configured for superposing the continuous linear motion of the piston by a mechanical displacement of the piston independent of the basal rate.

Embodiment 2: The drive system according to the preceding embodiment, wherein the mechanical displacement of the piston independent of the basal rate defines a bolus of insulin delivery.

Embodiment 3: The drive system according to any one of the preceding embodiments, further comprising at least one energy source configured for providing energy to the motor.

Embodiment 4: The drive system according to the preceding embodiment, wherein the energy source comprises at least one electrical energy source, more specifically at least one of a battery or an accumulator.

Embodiment 5: The drive system according to any one of the preceding embodiments, wherein the motor comprises at least one motor selected from the group consisting of: an electrical motor; a clockwork, preferably a spring-driven clockwork.

Embodiment 6: The drive system according to any one of the preceding embodiments, wherein the piston is mechanically coupled to a threaded rod.

Embodiment 7: The drive system according to the preceding embodiment, wherein the motor is coupled to the threaded rod via at least one gear, specifically via at least one gear comprised by the gear box.

Embodiment 8: The drive system according to the preceding embodiment, wherein the gear is mounted concentrically about the threaded rod.

Embodiment 9: The drive system according to any one of the three preceding embodiments, wherein the threaded rod is secured against rotation about its axis, preferably by at least one of a bolt or a toggle.

Embodiment 10: The drive system according to any one of the three preceding embodiments, wherein the gear is mounted to the threaded rod by a coupling, the coupling having at least two engagement elements capable of engaging with the threaded rod at at least two axially displaced engagement positions.

Embodiment 11: The drive system according to the preceding embodiment, wherein the engagement elements each comprise at least one ratchet, wherein, specifically, the engagement elements form a double-ratchet arrangement.

Embodiment 12: The drive system according to any one of the two preceding embodiments, wherein the engagement elements each comprise a rigid base surrounding the threaded rod and ratchet arms extending in an axial direction from the rigid base, wherein the ratchet arms are configured to engage with at least one thread of the threaded rod.

Embodiment 13: The drive system according to the preceding embodiment, wherein the ratchet arms extend from the rigid base in a direction towards the piston.

Embodiment 14: The drive system according to any one of the four preceding embodiments, wherein the engagement elements are mounted on the threaded rod in a fashion that the engagement elements are shiftable with respect to one another.

Embodiment 15: The drive system according to the preceding embodiment, wherein the engagement elements are connected via at least two bearing rods, wherein at least one of the engagement elements is mounted on the bearing rods in an axially shiftable fashion.

Embodiment 16: The drive system according to any one of the six preceding embodiments, wherein the engagement elements are connected via at least one axially acting spring element.

Embodiment 17: The drive system according to any one of the seven preceding embodiments, wherein the engagement elements are connected in a rotationally fixed fashion.

Embodiment 18: The drive system according to any one of the eight preceding embodiments, wherein at least three coupling states are adoptable by the coupling:
a first state, wherein in the first state both engagement elements engage with the threaded rod, wherein the threaded rod is driven in an axial direction by a rotation of the gear about an axis of the threaded rod, wherein in the first state, the engagement elements are located at a fixed spatial separation from each other;
a second state, wherein in the second state a first engagement of the engagement elements engages with the threaded rod and a second engagement of the engagement elements disengages with the threaded rod, wherein the first engagement element pushes the threaded rod in an axial direction through the second engagement element in a first axial direction towards the piston; and
a third state, wherein the first engagement element disengages with the threaded rod and the second engagement element engages with the threaded rod, wherein in the third state the first engagement element is pushed back in a second axial direction opposing the first axial direction.

Embodiment 19: The drive system according to the preceding embodiment, wherein the mechanical displacement of the piston independent of the basal rate is performed in the second state.

Embodiment 20: The drive system according to any one of the nine preceding embodiments, wherein the gear is driven by the motor via a drive, wherein the gear, via the coupling, drives the threaded rod.

Embodiment 21: The drive system according to any one of the ten preceding embodiments, wherein the gear is clamped between at least one of the engagement elements and a bushing.

Embodiment 22: The drive system according to any one of the eleven preceding embodiments, wherein the drive system further comprises at least one spring abutment, concentrically mounted about the threaded rod and configured for limiting a movement of the coupling in a direction away from the piston.

Embodiment 23: The drive system according to any one of the preceding embodiments, wherein the mechanical displacement unit comprises at least one displacement lever configured for one or both of exerting an axial pressure onto the piston, specifically via the threaded rod, more specifically via the threaded rod onto the piston, or axially displacing the piston, specifically via the threaded rod.

Embodiment 24: The drive system according to any one of the four preceding embodiments, wherein the drive comprises at least one gearwheel, preferably more than one gearwheel, for adjusting a transmission ratio between the motor and the gear.

Embodiment 25: The drive system according to the preceding embodiment, wherein the drive comprises at least one belt for transmitting the rotation of the motor onto the gear.

Embodiment 26: The drive system according to any one of the two preceding embodiments, wherein the drive comprises at least one endless screw configured for transmitting and/or converting the rotation of the at least one gearwheel onto the gear in an orthogonal fashion.

Embodiment 27: The drive system according to any one of the three preceding embodiments, wherein the belt interacts with a first gearwheel thereby transmitting the rotation of the motor onto the first gearwheel, wherein the first gearwheel interacts with a second gearwheel, wherein the second gearwheel further interacts with a third gearwheel, wherein the third gearwheel further interacts with a fourth gearwheel, wherein the fourth gearwheel further interacts with a fifth gearwheel, wherein the fifth gearwheel is connected to the endless screw, wherein the endless screw further interacts with the gear, wherein a rotation axis of the endless screw is arranged orthogonally to a rotation axis of the gear.

Embodiment 28: The drive system according to any one of the preceding embodiments, wherein the drive system further comprises a mechanical occlusion detection system.

Embodiment 29: An insulin pump for delivering insulin to a user, comprising:
at least one insulin reservoir; and
at least one drive system according to any one of the preceding embodiments referring to a drive system.

Embodiment 30: The insulin pump according to the preceding embodiment, wherein the insulin pump further comprises a housing.

Embodiment 31: The insulin pump according to any one of the two the preceding embodiments, wherein the insulin pump further comprises a starting element configured for starting delivering insulin using the insulin pump.

Embodiment 32: A method for driving an insulin pump, the method comprising:
a) rotating a motor at a predetermined revolution speed;
b) converting a rotation of the motor into a continuous linear motion of a piston by using a gear box, wherein the continuous linear motion of the piston determines a basal rate of insulin delivery; and
c) superposing the continuous linear motion of the piston by a mechanical displacement of the piston independent of the basal rate, by using a mechanical displacement unit.

Embodiment 33: The method according to the preceding embodiment, wherein the method further comprises delivering a bolus of insulin, the bolus of insulin being defined by the mechanical displacement of the piston independent of the basal rate.

Embodiment 34: The method according to any one of the preceding method embodiments, wherein the method further comprises providing energy, specifically electrical energy, to the motor by using at least one energy source.

Embodiment 35: The method according to the preceding embodiment, wherein the method comprises providing electrical energy to the motor by using at least one of a battery or an accumulator.

Embodiment 36: The method according to any one of the preceding method embodiments, wherein the motor comprises at least one motor selected from the group consisting of: an electrical motor; a clockwork, preferably a spring-driven clockwork.

Embodiment 37: The method according to any one of the preceding method embodiments, wherein method step b) further comprises mechanically coupling the piston to a threaded rod.

Embodiment 38: The method according to the preceding embodiment, wherein method step b) further comprises coupling the motor to the threaded rod via at least one gear.

Embodiment 39: The method according to the preceding embodiment, wherein method step b) further comprises mounting the gear concentrically about the threaded rod.

Embodiment 40: The method according to any one of the three preceding embodiments, wherein method step b) further comprises securing the threaded rod against rotation about its axis, preferably by using at least one of a bolt or a toggle.

Embodiment 41: The method according to any one of the three preceding embodiments, wherein method step b) comprises mounting the gear to the threaded rod by a coupling, the coupling having at least two engagement elements capable of engaging with the threaded rod at at least two axially displaced engagement positions.

Embodiment 42: The method according to the preceding embodiment, wherein the engagement elements each comprise at least one ratchet.

Embodiment 43: The method according to any one of the two preceding embodiments, wherein the engagement elements each comprise a rigid base surrounding the threaded rod and ratchet arms extending in an axial direction from the rigid base, wherein method step b) further comprises engaging the ratchet arms with at least one thread of the threaded rod.

Embodiment 44: The method according to the preceding embodiment, wherein the ratchet arms extend from the rigid base in a direction towards the piston.

Embodiment 45: The method according to any one of the four preceding embodiments, wherein the engagement element is mounted on the threaded rod in a fashion that the engagement elements are shiftable with respect to one another.

Embodiment 46: The method according to the preceding embodiment, wherein the engagement elements are connected via at least two bearing rods, wherein at least one of the engagement elements is mounted on the bearing rods in an axially shiftable fashion.

Embodiment 47: The method according any one of the six preceding embodiments, wherein the engagement elements are connected via at least one axially acting spring element.

Embodiment 48: The method according to any one of the seven preceding embodiments, wherein the engagement elements are connected in a rotationally fixed fashion.

Embodiment 49: The method according to any one of the eight preceding embodiments, wherein at least three coupling states are adoptable by the coupling:
a first state, wherein in the first state both engagement elements engage with the threaded rod, wherein the threaded rod is driven in an axial direction by a rotation of the gear about an axis of the threaded rod, wherein in the first state, the engagement elements are located at a fixed spatial separation from each other;
a second state, wherein in the second state a first engagement of the engagement elements engages with the threaded rod and a second engagement of the engagement elements disengages with the threaded rod, wherein the first engagement element pushes the threaded rod in an axial direction through the second engagement element in a first axial direction towards the piston; and
a third state, wherein the first engagement element disengages with the threaded rod and the second engagement element engages with the threaded rod, wherein in the third state the first engagement element is pushed back in a second axial direction opposing the first axial direction.

Embodiment 50: The method according to the preceding embodiment, wherein step c) comprises the second state.

Embodiment 51: The method according to any one of the nine preceding embodiments, wherein step b) further comprises driving the gear by the motor via a drive, wherein step b) further comprises driving the threaded rod by the gear via the coupling.

Embodiment 52: The method according to any one of the ten preceding embodiments, wherein step b) further comprises clamping the gear between at least one of the engagement elements and a bushing.

Embodiment 53: The method according to any one of the eleven preceding embodiments, wherein step b) further comprises mounting at least one spring abutment concentrically about the threaded rod and via the spring abutment limiting a movement of the coupling in a direction away from the piston.

Embodiment 54: The method according to any one of the preceding embodiments, wherein step c) further comprises one or both of exerting an axial pressure onto the piston, specifically via the threaded rod, more specifically via the threaded rod onto the piston, or axially displacing the piston, specifically via the threaded rod, by a displacement lever, wherein the displacement lever is comprised by the mechanical displacement unit.

Embodiment 55: The method according to any one of the four preceding embodiments, wherein step b) comprises adjusting a transmission ratio between the motor and the gear by using at least one gearwheel, preferably more than one gearwheel, comprised by the drive.

Embodiment 56: The method according to the preceding embodiment, wherein step b) further comprises transmitting the rotation of the motor onto the gear by using at least one belt comprised by the drive.

Embodiment 57: The method according to any one of the two preceding embodiments, wherein step b) further comprises converting the rotation of the at least one gearwheel onto the gear in an orthogonal fashion by using at least one endless screw.

Embodiment 58: The method according to any one of the three preceding embodiments, wherein step b) comprises:
b1) transmitting the rotation of the motor onto the at least one gearwheel;
b2) transmitting the rotation of the gearwheel onto the endless screw; and
b3) transmitting the rotation of the endless screw onto the gear, such that an axis of rotation of the endless screw is arranged orthogonally to an axis of rotation of the gear.

Embodiment 59: The method according to the preceding embodiment, wherein step b1) comprises transmitting the rotation of the motor onto a first gearwheel of a chain of gearwheels comprising at least two gearwheels, preferably three gearwheels, more preferably four gearwheels and wherein step b2) comprises transmitting the rotation of the last gearwheel of the chain of gearwheels onto the endless screw.

Embodiment 60: The method according to any one of the preceding method embodiments, wherein the method further comprises detecting an occlusion by using a mechanical occlusion detection system.

### BRIEF DESCRIPTION OF THE DRAWINGS

The above-mentioned aspects of exemplary embodiments will become more apparent and will be better understood by reference to the following description of the embodiments taken in conjunction with the accompanying drawings, wherein:

FIGS. 1A and 1B illustrate embodiments of an insulin pump in a perspective view;

FIG. 2 illustrates an embodiment of a drive system in a perspective view;

FIG. 3 illustrates an embodiment of a drive system in a top plan view;

FIG. 4 illustrates a sectional view of part of an embodiment of a drive system;

FIG. 5 illustrates a sectional view of part of an embodiment of a drive system; and

FIG. 6 illustrates a flow chart of an embodiment of a method for driving an insulin pump.

### DESCRIPTION

The embodiments described below are not intended to be exhaustive or to limit the invention to the precise forms disclosed in the following detailed description. Rather, the embodiments are chosen and described so that others skilled in the art may appreciate and understand the principles and practices of this disclosure.

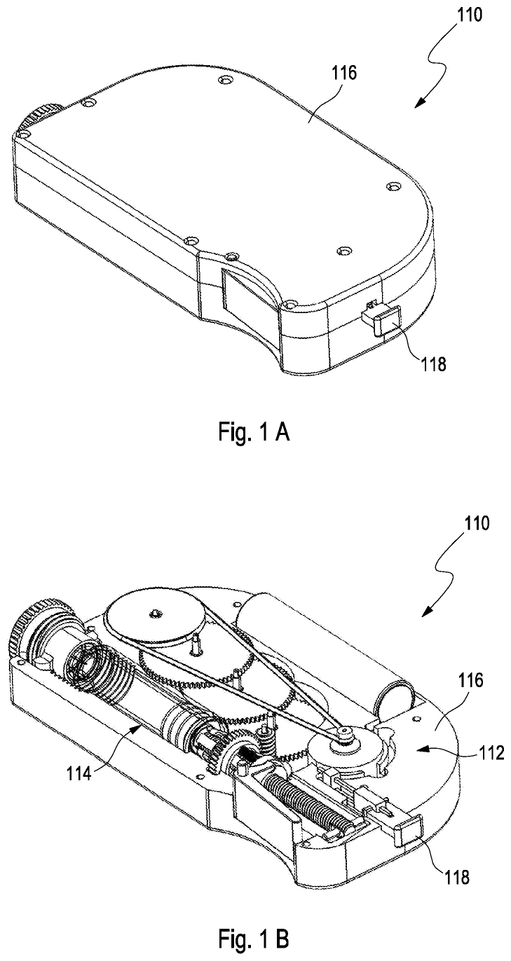

FIGS. 1A and 1B illustrate embodiments of an insulin pump 110 in a perspective view. The insulin pump 110 comprises a drive system (also referred to as a “drive”) 112 and an insulin reservoir 114. The insulin pump may further comprise a housing 116, wherein the housing may, for example, comprise two separate housing parts configured to fully or partially enclose the drive system 112 and the insulin reservoir 114. Further, as illustrated in FIGS. 1A and B, the housing may comprise a starting element (also referred to as a “starter”) 118 configured for starting delivering insulin using the insulin pump 110.

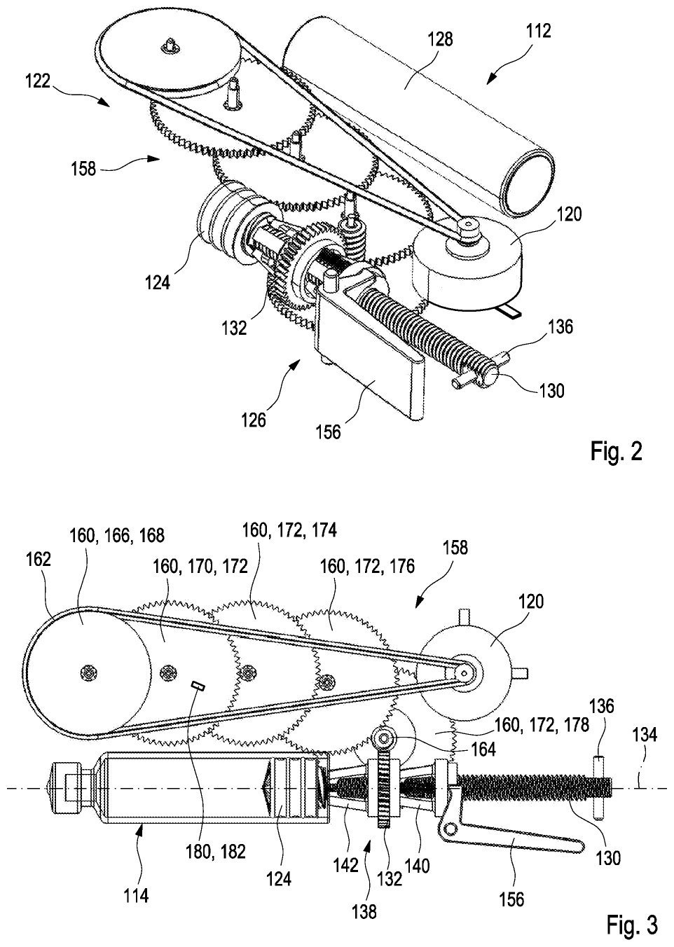

FIG. 2 illustrates an embodiment of a drive system 112 in a perspective view. The drive system comprises a motor 120 configured for rotating at a predetermined revolution speed. Additionally, the motor comprises a gear box 122 for converting a rotation of the motor 120 into a continuous linear motion of a piston 124. The continuous linear motion of the piston 124 determines a basal rate of insulin delivery. Further, the drive system comprises a mechanical displacement unit 126 (also referred to as “mechanical displacer”) configured for superposing the continuous linear motion of the piston 124 by a mechanical displacement of the piston 124 independent of the basal rate. Further, the drive system 112 may comprise an energy source 128, such as, for example, a battery or an accumulator, configured for providing energy to the motor 120. The motor 120 may, for example, be an electrical motor.

The piston 124 may specifically be mechanically coupled to a threaded rod 130. Further, the motor 120 may be coupled to the threaded rod 130 via a gear 132. As illustrated, the gear 132 may be mounted concentrically about the threaded rod 130. The threaded rod 130 may be secured against rotation about its axis 134, as illustrated in FIG. 3. Specifically, the threaded rod 130 may be secured against rotation about its axis 134 by a toggle 136, as illustrated in FIGS. 2 and 3.

The gear 132 may be mounted to the threaded rod 130 by a coupling 138, wherein the coupling 138 may have a first engagement element 140 and a second engagement element 142, wherein the engagement elements 140,142 may be capable of engaging with the threaded rod 130 at two axially displaced engagement positions. The engagement elements 140, 142 of the coupling 138, as illustrated in FIG. 5, may each comprise at least one ratchet. Specifically, the engagement elements 140, 142 may each comprise a rigid base 144 surrounding the threaded rod 130. The engagement elements 140, 142 further may each comprise ratchet arms 146 extending in an axial direction from the rigid base 144. In particular, the ratchet arms 146 may extend from the rigid base 144 in a direction towards the piston 124. The ratchet arms 146 may specifically be configured for engaging with at least one thread of the threaded rod 130, as illustrated in FIG. 5. The engagement elements 140,142 may further be mounted on the threaded rod 130 in a fashion that the engagement elements 140, 142 are shiftable with respect to one another.

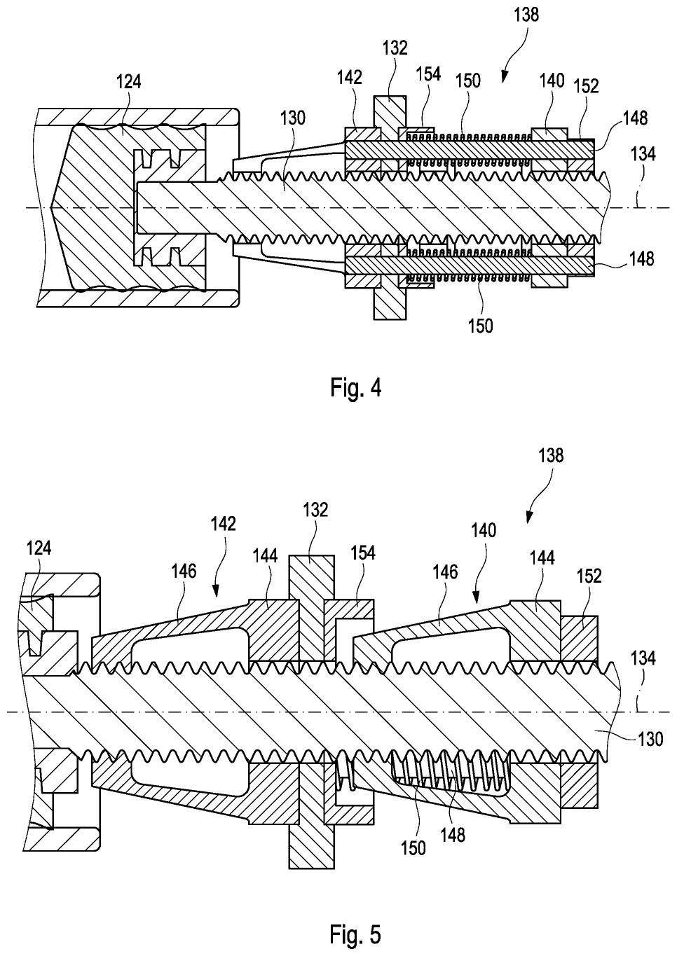

As illustrated in FIG. 4, the engagement elements 140, 142 may be connected via two bearing rods 148. Further, the engagement elements 140, 142 may be mounted on the bearing rods 148 in a fashion that at least one of the engagement elements 140, 142 may be shiftable on the bearing rods 148. In particular, the engagement elements 140, 142 may be connected via the bearing rods 148 such that the engagement elements 140, 142 may be rotationally fixed to each other. The engagement elements 140, 142 may further be connected via two axially acting spring elements (“springs”) 150. Specifically, the drive system may further comprise a spring abutment 152. The spring abutment 152 may limit a movement of the spring elements 150. Further, the spring abutment 152 may be configured for limiting a movement of the coupling 138 in a direction away from the piston 124. As further illustrated in FIGS. 4 and 5, the gear 132 may be clamped between the second engagement element 142 and a bushing 154.

The coupling 138 may comprise three coupling states. In particular, three coupling states may be adoptable by the coupling 138. Specifically, in a first coupling state the first engagement element 140 and the second engagement element 142 may engage with the threaded rod 130. In particular, in the first coupling state the two engagement elements 140, 142 may be located at a fixed spatial separation from each other on the threaded rod 130. In the first state the threaded rod 130, specifically the piston 124 connected to the threaded rod 130, may be driven in an axial direction by a rotation of the gear 132 about the axis 134 of the threaded rod 130. Specifically, in the first coupling state only the basal rate may be delivered to the user by the insulin pump 110.

In a second coupling state, the first engagement element 140 may still be engaged with the threaded rod 130, wherein the second engagement element 142 may disengage with the threaded rod 130. In particular, the first engagement element 140 may push the threaded rod 130 in an axial direction, specifically along the axis 134 of the threaded rod 130, through the second engagement element 142. Specifically, in the second coupling state the first engagement element 140 may push the threaded rod 130 through the second engagement element 142 in a first axial direction towards the piston 124. Thus, in the second coupling state both basal rate and bolus may be delivered to the user by the insulin pump 110. In particular, in the second state, the threaded rod 130, specifically the piston 124 connected to the threaded rod 130, may be driven in an axial direction by a rotation of the gear 132 about the axis 134, wherein the rotation of the gear 132 may drive the threaded rod 130 via the second engagement element 142. Specifically, the movement or part of the movement of the piston 124 powered or driven by the rotation of the gear 132 may lead to a basal rate delivering of insulin to the user. Additionally, in the second state, the piston 124 may be moved in an axial direction by the first engagement element 140 pushing the threaded rod 130. In particular, the additional movement of the piston 124 may lead to an additional insulin delivery to the user, for example, to a bolus of insulin. Thus, the delivering of the additional insulin, for example, the bolus, may be added to the basal rate delivery of insulin in the second state. Specifically, the basal rate of insulin may be superposed by the bolus. In particular, the mechanical displacement of the piston 124 independent of the basal rate may be performed in the second state.

In the third coupling state, the second engagement element 142 may engage with the threaded rod 130 and the first engagement element 140 may disengage with the threaded rod 130. Specifically, the first engagement element 140 may be pushed back in a second axial direction opposing the first axial direction. For example, the first engagement element 140 may be pushed back by the spring element 150. Thus, after the third coupling state, the coupling may switch to the first coupling state. As an example, the coupling 138 may be configured to switch from the first coupling state to the second coupling state and from the second coupling state to the third coupling state, preferably repeatedly.

As further illustrated in FIGS. 2 and 3, the mechanical displacement unit 126 may comprise a displacement lever 156. In particular, the displacement lever 156 may be configured for one or both of exerting an axial pressure onto the piston 124 or axially displacing the piston 124 when activated. Specifically, the activation of the displacement lever 156, for example, manually pushing the displacement lever 156, may exert an axial pressure onto the piston 124 via the threaded rod 130, more specifically via the first engagement element 140 onto the threaded rod 130 and via the threaded rod 130 onto the piston 124. As an example, the displacement lever 156 may be configured for triggering or pushing the coupling 138, specifically the first engagement element 140, such that the coupling switches from the first coupling state to the second coupling state, from the second coupling state to the third coupling state and from the third coupling state back to the first coupling state. Thus, the displacement lever 156 may allow delivering the bolus to the user, preferably by repeatedly activating the displacement lever 156.

Further, the drive system 112 may comprise a drive 158. In particular, the gear 132 may be driven by the motor 120 via the drive 158. The drive 158 may, for example, be part of the gear box 122. In particular, the drive 158 may comprise a plurality of gearwheels 160 for adjusting a transmission ratio between the motor 120 and the gear 132. Further, the drive 158 may comprise a belt 162 for transmitting the rotation of the motor 120 onto the gear 132, specifically via the gearwheels 160 onto the gear 132. Additionally, the drive 158 may comprise an endless screw 164 for converting the rotation of the gearwheels 160 onto the gear 132 in an orthogonal fashion. As illustrated in FIGS. 2 and 3, the drive may comprise five gearwheels 160. A first gearwheel 166 may, for example, comprise a friction wheel 168 interacting with the belt 162 and a spur wheel (not shown) interacting with a second gearwheel 170. The second gearwheel 170 may comprise a toothed gearwheel 172 and a spur wheel (not shown) for interacting with a third gearwheel 174. The third gearwheel 174 may, for example, comprise a toothed gearwheel 172 and a spur wheel (not shown) for interacting with a fourth gearwheel 176, wherein the fourth gearwheel 176 may comprise a toothed gearwheel 172 and a spur wheel (not shown) for interacting with a fifth gearwheel 178. As an example, the fifth gearwheel 178 may be connected to the endless screw 164.

Further, the drive system may comprise a mechanical occlusion detection system 180. In particular, the mechanical occlusion detection system 180 may, for example, be configured for providing information on an unwanted occlusion or blockage in a flow path of insulin. In particular, the mechanical occlusion detection system 180 as illustrated in FIG. 3 may provide a visual information to the user by providing a visually detectable mark 182 on the second gearwheel 170. In particular, the user may be able to identify an occurring occlusion when the second gearwheel 170 does not perform a rotation, e.g., by visually inspecting the detectable mark 182 through a viewing widow in the housing 116.

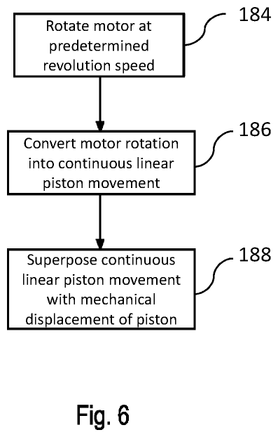

FIG. 6 illustrates a flow chart of an embodiment of a method for driving an insulin pump. The method comprises step a) (method step 184) of rotating a motor 120 at a predetermined revolution speed. Specifically, the motor 120 as illustrated in FIGS. 2 and 3 may be rotated in method step 184.

Further, the method comprises step b) (method step 186) of converting a rotation of the motor 120 into a continuous linear motion of a piston 124 by using a gear box 122, wherein the continuous linear motion of the piston 124 determines a basal rate of insulin delivery. Specifically, in method step 186 the rotation of the motor 120 may be converted into the continuous liner motion of the piston 124 by using the gear box 122, as illustrated in FIGS. 2 and 3.

The method further comprises step c) (method step 188) of superposing the continuous linear motion of the piston 124 by a mechanical displacement of the piston 124 independent of the basal rate, by using a mechanical displacement unit 126. In particular, the continuous linear motion of the piston 124 may be superposed by the mechanical displacement of the piston 124, for example, by activating the displacement lever 156 as illustrated in FIGS. 2 and 3.

While exemplary embodiments have been disclosed hereinabove, the present invention is not limited to the disclosed embodiments. Instead, this application is intended to cover any variations, uses, or adaptations of this disclosure using its general principles. Further, this application is intended to cover such departures from the present disclosure as come within known or customary practice in the art to which this invention pertains and which fall within the limits of the appended claims.

### LIST OF REFERENCE NUMBERS

110 insulin pump
112 drive system
114 insulin reservoir
116 housing
118 starting element
120 motor
122 gear box
124 piston
126 mechanical displacement unit
128 energy source
130 threaded rod
132 gear
134 axis
136 toggle
138 coupling
140 first engagement element
142 second engagement element
144 rigid base
146 ratchet arms
148 bearing rod
150 spring element
152 spring abutment
154 bushing
156 displacement lever
158 drive
160 gearwheel
162 belt
164 endless screw
166 first gearwheel
168 friction wheel
170 second gearwheel
172 toothed gearwheel
174 third gearwheel
176 fourth gearwheel
178 fifth gearwheel
180 mechanical occlusion detection system
182 visually detectable mark
184 step a): rotating a motor at a predetermined revolution speed
186 step b): converting a rotation of the motor into a continuous linear motion of a piston
188 step c): superposing the continuous linear motion of the piston by a mechanical displacement of the piston independent of the basal rate

What is claimed is:

1. A drive system for an insulin pump, comprising:
a motor configured to rotate at a predetermined revolution speed;
a gear box configured for acting on a piston of the insulin pump to thereby convert a rotation of the motor into a continuous linear motion of the piston via a threaded rod mechanically coupled to the piston, wherein the continuous linear motion of the piston determines a basal rate of insulin delivery; and
a mechanical displacer configured to act on the piston by adding a mechanical displacement of the threaded rod to the continuous linear motion of the piston, wherein the threaded rod is mechanically coupled to the piston independent of the basal rate.

2. The drive system according to claim 1, wherein the threaded rod has an axis of rotation and the threaded rod is secured against rotation about said axis.

3. The drive system according to claim 1, wherein the motor is coupled to the threaded rod via a gear mounted concentrically about the threaded rod.

4. The drive system according to claim 3, wherein the gear is mounted to the threaded rod by a coupling having at least two engagement elements configured for engaging with the threaded rod at two axially displaced engagement positions.

5. The drive system according to claim 4, wherein the engagement elements each comprise at least one ratchet.

6. The drive system according to claim 4, wherein the engagement elements each comprise a rigid base surrounding the threaded rod and ratchet arms extending in an axial direction from the rigid base, wherein the ratchet arms are configured to engage with at least one thread of the threaded rod.

7. The drive system according to claim 4, wherein the engagement elements are mounted on the threaded rod such that the engagement elements are shiftable axially relative to one another.

8. The drive system according to claim 7, wherein the engagement elements are connected via bearing rods, wherein at least one of the engagement elements is mounted on the bearing rods in an axially shiftable fashion.

9. The drive system according to claim 3, wherein the engagement elements are connected via at least one axially acting spring.

10. The drive system according to claim 3, wherein the engagement elements are rotationally fixed relative to one another.

11. The drive system according to claim 3, wherein:
the coupling has a first state in which both engagement elements engage with the threaded rod and the threaded rod is driven in an axial direction by a rotation of the gear about an axis of the threaded rod, wherein in the first state, the engagement elements are located at a fixed spacing from each other;
the coupling has a second state in which a first engagement of the engagement elements engages with the threaded rod and a second engagement of the engagement elements disengages with the threaded rod, wherein the first engagement element pushes the threaded rod in an axial direction through the second engagement element in a first axial direction towards the piston; and
the coupling has a third state in which the first engagement element disengages with the threaded rod and the second engagement element engages with the threaded rod, wherein in the third state the first engagement element is pushed back in a second axial direction opposing the first axial direction.

12. The drive system according to claim 1, wherein the mechanical displacer comprises at least one displacement lever configured for one or both of exerting an axial pressure onto the piston or axially displacing the piston.

13. An insulin pump for delivering insulin to a user, comprising:
at least one insulin reservoir; and
at least one drive system according to claim 1.

14. A method for driving an insulin pump, the method comprising:
a) rotating a motor at a predetermined revolution speed;
b) converting the rotation of the motor into a continuous linear motion of a piston via a threaded rod mechanically coupled to the piston by using a gear box, wherein the continuous linear motion of the piston determines a basal rate of insulin delivery; and
c) using a mechanical displacer to add a mechanical displacement of the threaded rod and thereby adding a displacement of the piston to the continuous linear motion of the piston, wherein the threaded rod is mechanically coupled to the piston independent of the basal rate.

15. The method according to claim 14, wherein step b) comprises:
b1) transmitting the rotation of the motor onto at least one gearwheel;
b2) transmitting the rotation of the gearwheel onto an endless screw; and
b3) transmitting the rotation of the endless screw onto the gear such that an axis of rotation of the endless screw is arranged orthogonally to an axis of rotation of the gear.
